(19) United States
(12) Patent Application Publication
(10) Pub. No.: US 2007/0212395 A1
(43) Pub. Date: Sep. 13, 2007

(54) Ocular therapy using sirtuin-activating agents

(75) Inventors: John E. Donello, Dana Point, CA (US); Rong Yang, Mission Viejo, CA (US); Elizabeth WoldeMussie, Laguna Niguel, CA (US); Fabien J. Schweighoffer, Fontenay-Sous-Bois (FR)

Correspondence Address: Stephen Donovan;Allergan, Inc., 2525 Dupont Drive, Irvine, CA, 92612, US

(73) Assignee: ALLERGAN, INC., Irvine, CA (US)

(21) Appl. No.: 11/371,117

(22) Filed: Mar. 8, 2006

(51) Int. Cl. A61K 31/7048; A61K 31/353; A61F 2/02; A61K 31/05

(52) U.S. Cl. 424/426; 514/27; 514/456; 514/731

(57) ABSTRACT

Ophthalmically therapeutic compositions, such as polymeric drug delivery systems, include a therapeutic component that includes a sirtuin-activating agent, such as resveratrol, which, upon delivery to the posterior segment of a mammalian eye, treats ocular conditions. Methods of making and using the present compositions are also described.

### FIELD OF THE INVENTION

The present invention relates generally to therapeutically effective ophthalmic compositions, and methods of making and using such compositions. More particularly, the present invention relates to the use of one or more sirtuin-activating agents, such as resveratrol, for treating various ocular conditions in mammals.

### BACKGROUND

The mammalian eye is a complex organ comprising an outer covering including the sclera (the tough white portion of the exterior of the eye) and the cornea (the clear outer portion covering the pupil and iris). In a medial cross section, from anterior to posterior, the eye comprises features including, without limitation: the cornea, the anterior chamber (a hollow feature filled with a watery, clear fluid called the aqueous humor and bounded by the cornea in the front and the lens in the posterior direction), the iris (a curtain-like feature that can open and close in response to ambient light), the lens, the posterior chamber (filled with a viscous fluid called the vitreous humor), the retina (the innermost coating of the back of the eye and comprising light-sensitive neurons), the choroid (an intermediate layer providing blood vessels to the cells of the eye), and the sclera. The posterior chamber comprises approximately ⅔ of the inner volume of the eye, while the anterior chamber and its associated features (lens, iris etc.) comprise about ⅓ of the eye's inner volume.

Ophthalmic therapy is typically performed by topically administering compositions, such as eye drops, to the exterior surface of the eye. However, the delivery of therapeutic agents to the interior or back of the eye, or even the inner portions of the cornea, presents unique challenges. Drugs are available that may be used for treating diseases of the posterior segment of the eye, including pathologies of the posterior sclera, the uveal tract (located in the vascular middle layer of the eye, constituting the iris, ciliary body, and choroids), the vitreous, the choroid, the retina, and the optic nerve head (ONH).

However, a major limiting factor in the effective use of such agents is delivering the agent to the affected tissue. The urgency to develop such methods can be inferred from the fact that the leading causes of vision impairment and blindness are conditions linked to the posterior segment or the eye. These conditions include, without limitation, age-related ocular degenerative diseases such as, age-related macular degeneration (ARMD), proliferative vitreoretinopathy (PVR), retinal ocular condition, retinal damage, diabetic macular edema (DME), and endophthalmitis. Glaucoma, which is often thought of as a condition of the anterior chamber affecting the flow (and thus, the intraocular pressure (IOP)) of aqueous humor, also has a posterior segment component; indeed, certain forms of glaucoma are not characterized by high IOP, but mainly by retinal degeneration alone.

Thus, there remains a need for new delivery methods and systems for administering neuroprotective agents to a patient to treat ophthalmic conditions.

### SUMMARY

The present invention relates generally to the treatment of ocular or ophthalmic conditions or diseases, and relates particularly to the treatment of ocular conditions via ocular administration of one or more sirtuin-activating agents to the eye or eyes of a patient. Ocular administration of the sirtuin-activating agent or agents can provide a protective effect to retinal ganglion cells as well as other ocular cell types. The administration of such agents can successfully treat one or more ophthalmic conditions involving neurodegeneration and other cell degenerative conditions, as discussed herein.

Thus, the present invention encompasses ophthalmically compatible or ophthalmically acceptable compositions which comprise one or more sirtuin-activating agents. Such compositions can be in any form suitable for ocular administration. For example, the compositions may be suitable for intraocular administration. Such intraocular compositions can be administered into the eye without negatively affecting the properties of the eye, such as the optical properties or physiological properties of the eye. In certain embodiments, the compositions are intravitreal compositions, that is compositions suitable for intravitreal administration. The compositions can be liquid, semi-solid, or solid compositions, as discussed herein. The present invention also encompasses methods of making such compositions, and methods of using such compositions. For example, the present invention encompasses methods of treating an ophthalmic condition by administering the sirtuin-activating agent containing compositions to an eye of a patient, or the use of the present compositions in the treatment of one or more ophthalmic conditions. In addition, the present invention encompasses the use of a sirtuin-activating agent in the manufacture of a medicament, such as the present compositions, for the treatment of an ophthalmic condition, as described herein.

In at least one embodiment, the present compositions are implants. The present implants comprise an effective amount of a sirtuin-activating agent to treat an ophthalmic condition. The implants can release the sirtuin-activating agent in a therapeutically effective amount, such as a neuroprotective amount, for extended periods of time, such as for at least one week, at least one month, at least six months, or even for at least one year after placement in an eye of a patient in need of treatment. In an embodiment, the implant comprises an effective amount of resveratrol, salts thereof, or mixtures thereof.

Accordingly, an intraocular implant can comprise a sirtuin-activating agent, such as an SIRT1-activating agent; and a bioerodible polymer matrix that releases the sirtuin-activating agent at a rate effective to sustain release of an amount of the sirtuin-activating agent from the implant for at least about one week after the implant is placed in an eye.

In an embodiment, a method of making an intraocular implant comprises extruding a mixture of a sirtuin-activating agent and a bioerodible polymer component to form a bioerodible material that biodegrades or bioerodes at a rate effective to sustain release of an amount of the sirtuin-activating agent from the implant for at least about one week after the implant is placed in an eye.

In an embodiment, a method of treating an ocular condition comprises placing a bioerodible intraocular implant in an eye of an individual, the implant comprising a sirtuin-activating agent and a bioerodible polymer matrix, wherein the implant degrades or erodes at a rate effective to sustain release of an amount of the sirtuin-activating agent from the implant effective to treat the ocular condition of the individual.

Other embodiments include non-solid compositions which comprise one or more sirtuin-activating agents. For example, a viscous composition suitable for intravitreal administration may comprise a sirtuin-activating agent. One embodiment may be a composition which comprises hyaluronic acid and a sirtuin activating agent, such as resveratrol. Other embodiments may include liquid compositions, and still other embodiments may include compositions which solidify when placed in the eye. Methods of making and using these compositions are also encompassed by the present invention.

The present compositions and methods can be practiced to treat a condition of the posterior segment of a mammalian eye, such as a condition selected from the group consisting of macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigment epithelitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis and glaucoma comprises administering to the posterior segment of the eye a composition comprising an SIRT1-activating agent in an ophthalmically effective vehicle. Conditions treated with the present compositions and methods may be ophthalmic conditions involving ocular degeneration, such as neurodegeneration of retinal ganglion cells.

The compositions are administered to the eye using any suitable technique. For example, the compositions can be injected into the eye or can be surgically placed in the eye. For example, an implant may be placed in the eye using a trocar or similar instrument. The compositions deliver therapeutically effective amounts of the sirtuin-activating agents for prolonged periods of time. Therapeutic effects include the alleviation or reduction of one or more symptoms associated with the ophthalmic condition or conditions.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description, drawing, and claims.

### BRIEF DESCRIPTION OF THE DRAWINGS

FIG. 1 is a graph of retinal ganglion cell survival ratio (treated/control) as a function of resveratrol dose administered to subjects with optic nerve injury.

### DESCRIPTION

New therapeutic compositions and methods have been invented. The present compositions and methods provide therapeutically effective amounts of one or more sirtuin-activating agents to an eye of a patient. The compositions can release or deliver therapeutically effective amounts, such as neuroprotecting amounts, of the sirtuin-activating agent to the eye for prolonged periods of time to provide a desired therapeutic effect. Desirably, the sirtuin-activating agent is delivered to the retina of the eye to provide a protective effect to retinal ganglion cells, among others. Thus, the present compositions can reduce degeneration of retinal cells, such as retinal ganglion cells, and thereby treat one or more ophthalmic conditions.

The present compositions encompass intraocular implants, which may include a biodegradable component, a non-biodegradable component, and combinations thereof, as well as liquid and semi-solid compositions.

In one embodiment, an intraocular implant comprises a biodegradable polymer matrix. The biodegradable polymer matrix is one type of a drug release sustaining component. The biodegradable polymer matrix is effective in forming a biodegradable intraocular implant. The biodegradable intraocular implant comprises a sirtuin-activating agent associated with the biodegradable polymer matrix. The sirtuin-activating agent may be dispersed within the bioerodible polymer matrix. The matrix degrades at a rate effective to sustain release of an amount of the sirtuin-activating agent for a time greater than about one week (or one month, or any other suitable time) from the time in which the implant is placed in an ocular region or an ocular site, such as the vitreous of an eye. For example, wherein the sirtuin-activating agent is resveratrol, the matrix may release resveratrol at a rate effective to sustain release of a therapeutically effective amount of the resveratrol for a time from about two months to about six months.

Sirtuins are in a family of enzymes produced by almost all life forms, from single-celled organisms to plants to mammals. Sirtuins (silent information regulator proteins) are often produced in times of stress, such as famine. Sirtuins act as protector enzymes to protect cells and boost cellular survival.

A sirtuin found in yeast, SIR2, becomes activated when placed under stress. SIR2 increases deoxyribonucleic acid (DNA) stability and speeds cellular repairs. SIR2 also increases total cell lifespan. The human homolog, SIRT1, suppresses the p53 enzyme system normally involved in suppressing tumor growth and prompting cell death (apoptosis). By curbing p53 activity, SIRT1 prevents premature aging and apoptosis normally caused when cellular DNA is harmed or stressed, giving the cells enough time to repair any damage and averting unnecessary cell death.

The present invention relates to the use of sirtuin-activating agents or sirtuin-activating compounds (STACs) that are either selectively designed to possess the ability to be directed to tissue of the posterior segment of the eye, or which possess the ability, when administered to the posterior segment of the eye, to preferentially penetrate, be taken up by, and remain within the posterior segment of the eye, as compared to the anterior segment of the eye. More specifically, the invention is drawn to ophthalmic compositions and drug delivery systems that provide extended release of the sirtuin-activating agent to the posterior segment (or tissue comprising within the posterior segment) of an eye to which the agents are administered, and to methods of making and using such compositions and systems, for example, to treat or reduce one or more symptoms of an ocular condition to improve or maintain vision of a patient.

Several plant metabolites act as sirtuin-activating compounds (STACs). A variety of polyphenols activate STACs, such as resveratrol, quercetin (3,5,7,3′,4′-pentahydroxyflavone), butein (3,4,2′,4′-tetrahydroxychalcone), piceatannol (3,5,3′,4′-tetrahydroxy-trans-stilbene), isoliquiritigenin (4,2′,4′-trihydroxychalcone), fisetin (3,7,3′,4-tetrahydroxyflavone), other flavones, stilbenes, isoflavones, catechins, and tannins.

Resveratrol is found in the skins of young, unripe red grapes. Resveratrol is also found in eucalyptus, peanuts, blueberries, some pines (e.g., Scots pine and eastern white pine), Japanese knotweed (hu zhang in China), giant knotweed, and several other plants. Resveratrol naturally occurs in two related forms, or isomers, trans-resveratrol (3,5,4′-trihydroxy-trans-stilbene) and cis-resveratrol.

Resveratrol may be obtained commercially (typically as the trans isomer, e.g., from the Sigma Chemical Company, St. Louis, Mo. in the United States), or it may be isolated from plant sources (such as wine or grape skins), or it may be chemically synthesized. Synthesis is typically conducted by a Wittig reaction linking two substituted phenols via a styrene double bond, as described by Moreno-Manas et al. (1985) Anal. Quim. 81:157-61 and subsequently modified by others (Jeandet, et al. (1991) Am. J. Enol. Vilic. 42:41-46; Goldberg, et al. (1994) Anal. Chem. 66: 3959-63).

In part, the present invention is drawn to methods of treating a variety of conditions of the posterior segment including (without limitation): cystic macular edema, diabetic macular edema, diabetic retinopathy, uveitis, and wet macular degeneration, by the administration of sirtuin-activating agents, including resveratrol, to specifically target the tissue of the posterior segment of the eye. In other embodiments the invention is drawn to implants comprising such sirtuin-activating agents and to methods of administrating such sirtuin-activating agents.

In one embodiment a system comprising a sirtuin-activating agent is administered directly to the posterior segment by, for example, injection or surgical incision. In a further embodiment the system is injected directly into the vitreous humor in a fluid solution or suspension of crystals or amorphous particles comprising a sirtuin-activating agent. In another embodiment the system is comprises, consists essentially of, or consists only of an intravitreal implant. The sirtuin-activating agent may, without limitation, be provided in a reservoir of such implant, may be provided in a biodegradable implant matrix in such a manner that it is released as the matrix is degraded, or may be physically blended or mixed with the biodegradable polymeric matrix.

Additionally, a sirtuin-activating agent of the present invention may be administered to the posterior segment indirectly, such as (without limitation) by topical ocular administration, by subconjunctival, or subscleral injection. Such techniques may also require additional agents or method steps to provide a desired amount of the sirtuin-activating agent to the posterior of the eye, if desired.

The sirtuin-activating agents of the present invention all possess certain properties in accord with the present invention. First, the sirtuin-activating agent should have neuroprotective activities in brain ischemic models. Secondly, the sirtuin-activating agent should prolong cell life by activating sirtuin (presumably allosteric regulation of sirtuin). Finally, the sirtuin-activating agent should prevent axon degradation by activating SIRT1 in a mouse DRG culture model. Identification of such agents can be performed using any assay which analyzes these properties.

While a most preferred sirtuin-activating agent possesses all of these properties, a sirtuin-activating agent may possess less than all such properties so long as it possesses the property of remaining therapeutically active in the posterior chamber when delivered intravitreally.

Exemplary compounds that may be used in the present compositions and methods as sirtuin-activating agents are selected from the group consisting of flavones, stilbenes, flavanones, isoflavones, catechins, chalcones, tannins, anthocyanidins, and analogs and derivatives thereof. In illustrative embodiments, compounds are selected from the group consisting of resveratrol, butein, piceatannol, isoliquiritgenin, fisetin, luteolin, 3,6,3′4′-tetrahydroxyflavone, quercetin, and analogs and derivatives thereof. In addition, the agents may include either the cis or trans isomer of such compounds, and combinations thereof. For example, the agent may comprise approximately equal amounts of cis and trans isomers of such compounds, or the agent may comprise a major portion of the cis isomer or the trans isomer. In at least one specific embodiment, the agent is the trans-isomer of resveratrol. In certain embodiments, if the sirtuin-activating compound is a naturally occurring compound, it may not be in a form in which it is naturally occurring.

As described herein, controlled and sustained administration of a therapeutic agent through the use of one or more intraocular implants may improve treatment of undesirable ocular conditions. The implants comprise a pharmaceutically acceptable polymeric composition and are formulated to release one or more pharmaceutically active agents, such as sirtuin-activating agents or neuroprotective agents, including resveratrol, over an extended period of time. A sirtuin-activating agent may comprise at least one of resveratrol, derivatives thereof, salts thereof, isomers thereof, and mixtures thereof or other compounds described below. The implants are effective to provide a therapeutically effective dosage of the agent or agents directly to a region of the eye to treat, prevent, and/or reduce one or more symptoms of one or more undesirable ocular conditions. Thus, with a single administration, therapeutic agents will be made available at the site where they are needed and will be maintained for an extended period of time.

An intraocular implant in accordance with the disclosure herein comprises a therapeutic component and a drug release-sustaining component associated with the therapeutic component. In accordance with the present invention, the therapeutic component comprises, consists essentially of, or consists of, a sirtuin-activating agent or neuroprotective agent, such as resveratrol or the trans isomer of resveratrol. The drug release-sustaining component is associated with the therapeutic component to sustain release of an effective amount of the therapeutic component into an eye in which the implant is placed. The amount of the therapeutic component is released into the eye for a period of time greater than about one week after the implant is placed in the eye, and is effective in treating and/or reducing at least one symptom of one or more degenerative or neurodengerative ocular conditions, such as glaucoma, diabetic retinopathy, macular degeneration, and the like.

Definitions

For the purposes of this description, we use the following terms as defined in this section, unless the context of the word indicates a different meaning.

As used herein, an “intraocular implant” refers to a device or element that is structured, sized, or otherwise configured to be placed in an eye. Intraocular implants are generally biocompatible with physiological conditions of an eye and do not cause unacceptable adverse side effects. Intraocular implants may be placed in an eye without disrupting vision of the eye.

As used herein, a “therapeutic component” refers to a portion of an intraocular implant or other ophthalmic composition comprising one or more therapeutic agents or substances used to treat a medical condition of the eye. The therapeutic component may be a discrete region of an intraocular implant, or it may be homogenously distributed throughout the implant. The therapeutic agents of the therapeutic component are typically ophthalmically acceptable, and are provided in a form that does not cause adverse reactions when the implant or composition is placed in an eye.

As used herein, a “drug release-sustaining component” refers to a portion of the intraocular implant or composition that is effective to provide a sustained release of the therapeutic agents of the implant. A drug release-sustaining component may be a biodegradable polymer matrix, or it may be a coating covering a core region of the implant that comprises a therapeutic component.

As used herein, “associated with” means mixed with, dispersed within, coupled to, covering, or surrounding.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, an “ocular condition” is a disease, ailment, or condition that affects or involves the eye or one of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

An “anterior ocular condition” is a disease, ailment, or condition which affects or which involves an anterior (i.e., front of the eye) ocular region or site, such as a periocular muscle, an eye lid, or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e., reduce intraocular pressure (IOP)).

A “posterior ocular condition” is a disease, ailment, or condition which primarily affects or involves a posterior ocular region or site such as the choroid or the sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment, or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e., neuroprotection).

The term “biodegradable polymer” refers to a polymer or polymers which disintegrate or degrade in vivo, and wherein erosion of the polymer or polymers over time occurs concurrent with or subsequent to release of the therapeutic agent. Specifically, hydrogels such as methylcellulose, which act to release drug through polymer swelling, are specifically excluded from the term “biodegradable polymer”. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue.

The term “therapeutically effective amount” or “therapeutically effective concentration,” as used herein, refers to the level, amount, or concentration of agent needed to treat an ocular condition, or reduce, or prevent ocular injury or damage without causing significant negative or adverse side effects to the eye or a region of the eye or to improve at least one symptom of a disease, condition or disorder affecting an eye, as compared to an untreated eye. As discussed herein, in certain embodiments, a therapeutically effective amount” can be a neuroprotective amount of a sirtuin-activating agent.

As used herein, “periocular administration” refers to delivery of the therapeutic component to a retrobulbar region, a subconjunctival region, a subtenon region, a suprachoroidal region or space, and/or an intrascleral region or space. For example, a posterior directed sirtuin-activating agent may be associated with water, saline, a polymeric liquid or semi-solid carrier, phosphate buffer, or other ophthalmically acceptable liquid carrier. The present liquid-containing compositions are preferably in an injectable form. In other words, the compositions may be intraocularly administered, such as by intravitreal injection, using a syringe and needle or other similar device (e.g., see U.S. Patent Publication No. 2003/0060763), hereby incorporated by reference herein in its entirety, or the compositions can be periocularly administered using an injection device.

In part, the present invention is generally drawn to methods for treating the posterior segment of the eye. The posterior segment of the eye comprises, without limitation, the uveal tract, vitreous, retina, choroid, optic nerve, and the retinal pigmented epithelium (RPE). The disease or condition related to this invention may comprise any disease or condition that can be prevented or treated by the action of a sirtuin-activating agent, often resveratrol, including combinations such as resveratrol with quercetin, upon a posterior part of the eye. While not intending to limit the scope of this invention in any way, some examples of diseases or conditions that can be prevented or treated by the action of an active drug upon the posterior part of the eye in accordance with the present invention may include maculopathies/retinal degeneration such as macular edema, anterior uveitis, retinal vein occlusion, non-exudative age related macular degeneration, exudative age related macular degeneration (ARMD), choroidal neovascularization, diabetic retinopathy, acute macular neuroretinopathy, central serous chorioretinopathy, cystoid macular edema, and diabetic macular edema; uveitis/retinitis/choroiditis, such as acute multifocal placoid pigment epitheliopathy, Behcet's disease, birdshot retinochoroidopathy, infections (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; vascular diseases/exudative diseases such as retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders associated with retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis. Preferably, the disease or condition is retinitis pigmentosa, proliferative vitreal retinopathy (PVR), age-related macular degeneration (ARMD), diabetic retinopathy, diabetic macular edema, retinal detachment, retinal tear, uveitus, or cytomegalovirus retinitis. Glaucoma can also be considered a posterior ocular condition because the therapeutic goal is to prevent the loss of or reduce the occurrence of loss of vision due to damage to or loss of retinal cells or optic nerve cells (i.e. neuroprotection).

Such conditions may be treated by administering to the posterior segment of the eye a composition comprising a sirtuin-activating agent (e.g., a suspension of resveratrol particles) in an ophthalmically effective vehicle, such as a polymer (e.g., a bioerodible polymer). For example, the composition may comprise a polymeric component (e.g., comprising hyaluronic acid) administered intravitreally. The composition may comprise an intravitreal implant comprising a sirtuin-activating agent and a biocompatible polymer.

The bioerodible polymer of certain present implants may be selected from the group consisting of poly (lactide-co-glycolide) polymer (PLGA), poly-lacetic acid (PLA), poly-glycolic acid (PGA), polyesters, poly (ortho ester), poly (phosphazine), poly (phosphate ester), polycaprolactones, gelatin, and collagen, and derivatives and combinations thereof.

The present compositions include liquid-containing compositions (such as formulations) and polymeric drug delivery systems, among others. The present compositions may be understood to include solutions, suspensions, emulsions, and the like, such as other liquid-containing compositions used in ophthalmic therapies. Polymeric drug delivery systems comprise a polymeric component, and may be understood to include biodegradable implants, non-biodegradable implants, biodegradable microparticles, such as biodegradable microspheres, and the like. The present drug delivery systems may also be understood to encompass elements in the form of tablets, wafers, rods, sheets, filaments, sphere, particles, and the like. The polymeric drug delivery systems may be solid, semi-solid, or viscoelastic.

Particles are generally smaller than the implants disclosed herein, and may vary in shape. For example, certain embodiments of the present invention use substantially spherical particles. These particles may be understood to be microspheres. Other embodiments may utilize randomly configured particles, such as particles that have one or more flat or planar surfaces. The drug delivery system may comprise a population of such particles with a predetermined size distribution. For example, a major portion of the population may comprise particles having a desired diameter measurement. In another example, a sirtuin-activating agent may contain particles (such as particles comprising resveratrol) in solid form.

A sirtuin-activating agent (e.g., resveratrol or a trans isomer thereof) of the present methods and systems may be present in an amount in the range of about from about 40% by weight to about 70% by weight of the implant. The biodegradable polymer matrix may comprise a poly (lactide-co-glycolide) in an amount from about 30% by weight to about 60% by weight of the implant. The matrix may comprise at least one polymer selected from the group consisting of polylactides, poly (lactide-co-glycolides), derivatives thereof, and mixtures thereof. The matrix may be substantially free of polyvinyl alcohol, or in other words, includes no polyvinyl alcohol.

For intravitreally administered compounds, providing relatively high concentrations of the sirtuin-activating agent (for example, in the form of crystals or particles) may be beneficial in that reduced amounts of the compound may be required to be placed or injected into the posterior segment of the eye in order to provide the same amount or more of the therapeutic component in the posterior segment of the eye relative to other compositions.

In certain embodiments, the material further comprises a sirtuin-activating agent and an excipient component. The excipient component may be understood to include solubilizing agents, viscosity-inducing agents, buffer agents, tonicity agents, preservative agents, and the like.

In some embodiments of the present invention, a solubilizing agent may be a cyclodextrin. In other words, the present materials may comprise a cyclodextrin component provided in an amount from about 0.1% (w/v) to about 5% (w/v) of the composition. In further embodiments, the cyclodextrin comprises up to about 10% (w/v) of certain cyclodextrins, as discussed herein. In further embodiments, the cyclodextrin comprises up to about 60% (w/v) of certain cyclodextrins, as discussed herein. The excipient component of the present compositions may comprise one or more types of cyclodextrins or cyclodextrin derivatives, such as alpha-cyclodextrins, beta-cyclodextrins, gamma-cyclodextrins, and derivatives thereof. As understood by persons of ordinary skill in the art, cyclodextrin derivatives refer to any substituted or otherwise modified compound that has the characteristic chemical structure of a cyclodextrin sufficiently to function as a cyclodextrin, for example, to enhance the solubility and/or stability of therapeutic agents and/or reduce unwanted side effects of the therapeutic agents and/or to form inclusive complexes with the therapeutic agents.

Viscosity-inducing agents of the present invention, include without limitation, polymers that are effective in stabilizing the therapeutic component in the composition. The viscosity-inducing component is present in an effective amount for increasing the viscosity of the composition. Advantageously the viscosity-inducing component is present in an effective amount for substantially increasing the viscosity of the composition. Increased viscosities of the present compositions may enhance the ability of the present compositions to maintain the sirtuin-activating agent, including particles containing a sirtuin-activating agent, in substantially uniform suspension in the compositions for prolonged periods of time, for example, for at least about one week, without requiring resuspension processing.

The relatively high viscosity of certain of the present compositions may also have an additional benefit of at least assisting the compositions to have the ability to have an increased amount or concentration of the sirtuin-activating agent, as discussed elsewhere herein, for example, while maintaining such sirtuin-activating agent in substantially uniform suspension for prolonged periods of time.

The therapeutic compositions, including the sirtuin-activating agents described as part of the present invention, may be suspended in a viscous formulation having a relatively high viscosity, such as a viscosity approximating that of the vitreous humor. Such viscous formulation comprises a viscosity-inducing component. The therapeutic agent of the present invention may be administered intravitreally as, without limitation, an aqueous injection, a suspension, an emulsion, a solution, a gel, or inserted in a sustained release or extended release implant, either biodegradable or non-biodegradable.

The viscosity-inducing component preferably comprises a polymeric component and/or at least one viscoelastic agent, such as those materials that are useful in ophthalmic surgical procedures. Examples of useful viscosity-inducing components include, but are not limited to, hyaluronic acid, carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures thereof.

The molecular weight of the viscosity-inducing components may be in a range up to about 2 million Daltons, such as of about 10,000 Daltons or less to about 2 million Daltons or more. In one particularly useful embodiment, the molecular weight of the viscosity-inducing component is in a range of about 100,000 Daltons or about 200,000 Daltons to about 1 million Daltons or about 1.5 million Daltons.

In one very useful embodiment, a viscosity-inducing component is a polymeric hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component preferably is in a range of about 50,000 Daltons or about 100,000 Daltons to about 1.3 million Daltons or about 2 million Daltons.

In one embodiment, the sirtuin-activating agents of the present invention may be provided in a polymeric hyaluronate component in an amount in a range about 0.01% to about 0.5% (w/v) or more. In a further useful embodiment, the hyaluronate component is present in an amount in a range of about 1% to about 4% (w/v) of the composition. In this latter case, the very high polymer viscosity forms a gel that slows the sedimentation rate of any suspended drug, and prevents pooling of injected sirtuin-activating agent.

The sirtuin-activating agent of the present invention may include any or all salts, prodrugs, conjugates, analogs, derivatives, isomers, or precursors of such therapeutically useful sirtuin-activating agent, including those specifically identified herein.

In certain embodiments, the compositions of the present invention may comprise more than one ophthalmically acceptable therapeutic agent, so long as at least one such therapeutic agent is a sirtuin-activating agent having one or more of the properties described herein as important to treating ocular conditions. In other words, a therapeutic composition of the present invention, however administered, may include a first therapeutic agent, and one or more additional opthalmically acceptable therapeutic agents, or a combination of therapeutic agents, so long as at least one of such therapeutic agents is a sirtuin-activating agent.

Some specific examples of ophthalmically acceptable therapeutic agents include amantadine derivates, salts thereof, and combinations thereof. For example, the amantadine derivates may be memantine, amantadine, and rimantadine. Other antiexcitotoxic agents may include nitroglycerin, dextorphan, dextromethorphan, and CGS-19755. A notable ophthalmically acceptable therapeutic agent to combine with resveratrol is quercetin. One or more of the therapeutic agents in such compositions may be formed as or present in particles or crystals.

In these aspects of the present invention, the viscosity-inducing component is present in an effective amount to increase, advantageously substantially increase, the viscosity of the composition. Without wishing to limit the invention to any particular theory of operation, it is believed that increasing the viscosity of the compositions to values well in excess of the viscosity of water, for example, at least about 100 centipoises (cps) at a shear rate of 0.1/second, compositions which are highly effective for placement, e.g., injection, into the posterior segment of an eye of a human or animal are obtained. Along with the advantageous placement or injectability of the these compositions containing sirtuin-activating agents into the posterior segment, the relatively high viscosity of the present compositions are believed to enhance the ability of such compositions to maintain the therapeutic component (for example, comprising particles containing sirtuin-activating agents) in substantially uniform suspension in the compositions for prolonged periods of time, and may aid in the storage stability of the composition.

Advantageously, the compositions of this aspect of the invention may have viscosities of at least about 10 cps or at least about 100 cps or at least about 1000 cps, more preferably at least about 10,000 cps and still more preferably at least about 70,000 cps or more, for example up to about 200,000 cps or about 250,000 cps, or about 300,000 cps or more, at a shear rate of 0.1/second. In particular embodiments the present compositions not only have the relatively high viscosity noted above but also have the ability or are structured or made up so as to be effectively able to be placed, e.g., injected, into a posterior segment of an eye of a human or animal, for example, through a 27-gauge needle, or even through a 30 gauge needle.

The viscosity-inducing components preferably are shear thinning components such that as the viscous formulation is passed through or injected into the posterior segment of an eye, for example, through a narrow aperture, such as a 27-gauge needle. Under high shear conditions the viscosity of the composition is substantially reduced during such passage. After such passage, the composition regains substantially its pre-injection viscosity so as to maintain any particles, containing a sirtuin-activating agent, in suspension in the eye.

Any ophthalmically acceptable viscosity-inducing component may be employed in accordance with the sirtuin-activating agents in the present invention. Many such viscosity-inducing components have been proposed and/or used in ophthalmic compositions used on or in the eye. The viscosity-inducing component is present in an amount effective in providing the desired viscosity to the composition. Advantageously, the viscosity-inducing component is present in an amount in a range of about 0.5% or about 1.0% to about 5% or about 10% or about 20% (w/v) of the composition. The specific amount of the viscosity inducing component employed depends upon a number of factors including, for example and without limitation, the specific viscosity inducing component being employed, the molecular weight of the viscosity inducing component being employed, the viscosity desired for the composition, containing a sirtuin-activating agent, being produced and/or used and similar factors.

In another embodiment of the invention, the therapeutic agents (including at least one sirtuin-activating agent) may be delivered intraocularly in a composition that comprises, consists essentially of, or consists of, a therapeutic component comprising a sirtuin-activating agent and a biocompatible polymer suitable for administration to the posterior segment of an eye. For example, the composition may, without limitation, comprise an intraocular implant or a liquid or semisolid polymer. In another example, the implant is placed in the posterior segment of the eye (e.g., the implant is placed in the posterior of the eye with a trocar or syringe). Some intraocular implants are described in publications including U.S. Pat. Nos. 6,726,918; 6,699,493; 6,369,116; 6,331,313; 5,869,079; 5,824,072; 5,766,242; 5,632,984; and 5,443,505, these and all other publications cited or mentioned herein are hereby incorporated by reference herein in their entirety, unless expressly indicated otherwise. These are only examples of particular preferred implants, and others will be available to the person of ordinary skill in the art.

The polymer in combination with the therapeutic agent containing a sirtuin-activating agent may be understood to be a polymeric component. In some embodiments, the particles may comprise D,L-polylactide (PLA) or latex (carboxylate-modified polystyrene beads). In other embodiments the particles may comprise materials other than D,L-polylactide (PLA) or latex (carboxylate-modified polystyrene beads). In certain embodiments, the polymer component may comprise a polysaccharide. For example, the polymer component may comprise a mucopolysaccharide. In at least one specific embodiment, the polymer component is hyaluronic acid.

However, in additional embodiments, and regardless of the method of sirtuin-activating agent administration, the polymeric component may comprise any polymeric material useful in a body of a mammal, whether derived from a natural source or synthetic. Some additional examples of useful polymeric materials for the purposes of this invention include carbohydrate-based polymers such as methylcellulose, carboxymethylcellulose, hydroxymethylcellulose hydroxypropylcellulose, hydroxyethylcellulose, ethyl cellulose, dextrin, cyclodextrins, alginate, hyaluronic acid and chitosan, protein-based polymers such as gelatin, collagen and glycolproteins, and hydroxy acid polyesters such as bioerodible polylactide-coglycolide (PLGA), polylacetic acid (PLA), polyglycolide, polyhydroxybutyric acid, polycaprolactone, polyvalerolactone, polyphosphazene, and polyorthoesters. Polymers can also be cross-linked, blended, or used as copolymers in the invention. Other polymer carriers include albumin, polyanhydrides, polyethylene glycols, polyvinyl polyhydroxyalkyl methacrylates, pyrrolidone, and polyvinyl alcohol.

Some examples of non-erodible polymers include silicone, polycarbonates, polyvinyl chlorides, polyamides, polysulfones, polyvinyl acetates, polyurethane, ethylvinyl acetate derivatives, acrylic resins, cross-linked polyvinyl alcohol and cross-linked polyvinylpyrrolidone, polystyrene, and cellulose acetate derivatives.

These additional polymeric materials may be useful in a composition comprising the therapeutically useful sirtuin-activating agents disclosed herein, or for use in any of the methods, including those involving the intravitreal administration of such methods. For example, and without limitation, PLA or PLGA may be coupled to (or associated with) a sirtuin-activating agent for use in the present invention, either as particles in suspension, as part of an implant, or any other opthalmically suitable use. This insoluble conjugate will slowly erode over time, thereby continuously releasing the sirtuin-activating agent.

Regardless of the mode of administration or form (e.g., in solution, suspension, as a topical, injectable or implantable agent), the therapeutic compositions, containing one or more sirtuin-activating agents, of the present invention can be administered in a pharmaceutically acceptable vehicle component. The therapeutic agent or agents may also be combined with a pharmaceutically acceptable vehicle component in the manufacture of a composition. In other words, a composition, as disclosed herein, may comprise a therapeutic component and an effective amount of a pharmaceutically acceptable vehicle component. In at least one embodiment, the vehicle component is aqueous-based. For example, the composition may comprise water.

In certain embodiments, the therapeutic agent, containing a sirtuin-activating agent, is administered in a vehicle component, and may also include an effective amount of at least one of a viscosity-inducing component, a resuspension component, a preservative component, a tonicity component, and a buffer component. In some embodiments, the compositions disclosed herein include no added preservative component. In other embodiments, a composition may optionally include an added preservative component. In addition, the composition may be included with no resuspension component.

Formulations for topical or intraocular administration of the therapeutic component, containing a sirtuin-activating agent, (including, without limitation, implants or particles containing such agents) may include a major amount of liquid water (such as for a buffer component). Such compositions are preferably formulated in a sterile form, for example, before being used in the eye. The above-mentioned buffer component, if present in the intraocular formulations, is present in an amount effective to control the pH of the composition. The formulations may contain, either in addition to, or instead of, the buffer component at least one tonicity component in an amount effective to control the tonicity or osmolality of the compositions. Indeed, the same component may serve as both a buffer component and a tonicity component. More preferably, the present compositions include both a buffer component and a tonicity component.

The buffer component and/or tonicity component, if either is present, may be chosen from those that are conventional and well known in the ophthalmic art. Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, any other suitable ophthalmically acceptably tonicity component and mixtures thereof. Non-ionic tonicity components may comprise polyols derived from sugars, such as xylitol, sorbitol, mannitol, glycerol and the like.

The amount of buffer component employed preferably is sufficient to maintain the pH of the composition in a range of about 6 to about 8, more preferably about 7 to about 7.5. The amount of tonicity component employed preferably is sufficient to provide an osmolality to the present compositions in a range of about 200 to about 400, more preferably about 250 to about 350, mOsmol/kg respectively. Advantageously, the present compositions are substantially isotonic.

The compositions of, or used in, the present invention may include one or more other components in amounts effective to provide one or more useful properties and/or benefits to the present compositions. For example, although the present compositions may be substantially free of added preservative components, in other embodiments, the present compositions include effective amounts of preservative components, preferably such components that are more compatible with or friendly to the tissue in the posterior segment of the eye into which the composition is placed than benzyl alcohol. Examples of such preservative components include, without limitation, quaternary ammonium preservatives such as benzalkonium chloride (“BAC” or “BAK”) and polyoxamer; bigunanide preservatives such as polyhexamethylene biguandide (PHMB); methyl and ethyl parabens; hexetidine; chlorite components, such as stabilized chlorine dioxide, metal chlorites and the like; other ophthalmically acceptable preservatives and the like and mixtures thereof. The concentration of the preservative component, if any, in the present compositions is a concentration effective to preserve the composition, and (depending on the nature of the particular preservative used) is often and generally used in a range of about 0.00001% to about 0.05% (w/v) or about 0.1% (w/v) of the composition.

Other embodiments of the present compositions are in the form of a polymeric drug delivery system that is capable of providing sustained drug delivery for extended periods of time after a single administration. For example, the present drug delivery systems can release the sirtuin-activating agent for at least about 1 week, or about 1 month, or about 3 months, or about 6 months, or about 1 year, or about 5 years or more. Thus, such embodiments of the present invention may comprise a polymeric component associated with the therapeutic component in the form of a polymeric drug delivery system suitable for administration to a patient by at least one of intravitreal administration and periocular administration.

As discussed herein, the polymeric component of the present drug delivery systems may comprise a polymer selected from the group consisting of biodegradable polymers, non-biodegradable polymers, biodegradable copolymers, non-biodegradable copolymers, and combinations thereof. In certain embodiments, the polymeric component comprises a poly (lactide-co-glycolide) polymer (PLGA). In other embodiments, the polymeric component comprises a polymer (such as a bioerodible polymer matrix) selected from the group consisting of poly (lactide-co-glycolide) polymer (PLGA), poly-lacetic acid (PLA), poly-glycolic acid (PGA), polyesters, poly (ortho ester), poly (phosphazine), poly (phosphate ester), poly (D,L-lactide-co-glycolide), polyesters, polycaprolactones, gelatin, and collagen, and derivatives and combinations thereof. The polymeric component may be associated with the therapeutic component to form an implant selected from the group consisting of solid implants, semisolid implants, and viscoelastic implants.

The sirtuin-activating agent may be in a particulate or powder form and entrapped by a biodegradable polymer matrix. Usually, sirtuin-activating agent particles in intraocular implants will have an effective average size measuring less than about 3000 nanometers. However, in other embodiments, the particles may have an average maximum size greater than about 3000 nanometers. In certain implants, the particles may have an effective average particle size about an order of magnitude smaller than 3000 nanometers. For example, the particles may have an effective average particle size of less than about 500 nanometers. In additional implants, the particles may have an effective average particle size of less than about 400 nanometers, and in still further embodiments, a size less than about 200 nanometers. In addition, when such particles are combined with a polymeric component, the resulting polymeric intraocular particles may be used to provide a desired therapeutic effect.

If formulated as part of an implant or other drug delivery system, the sirtuin-activating agent of the present systems is preferably from about 1% to 90% by weight of the drug delivery system. More preferably, the sirtuin-activating agent is from about 20% to about 80% by weight of the system. In a preferred embodiment, the sirtuin-activating agent comprises about 40% by weight of the system (e.g., 30%-50%). In another embodiment, the sirtuin-activating agent comprises about 60% by weight of the system.

In addition to the foregoing, examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked.

For the most part, besides carbon and hydrogen, the polymers will include at least one of oxygen and nitrogen, advantageously oxygen. The oxygen may be present as oxy, e.g. hydroxy or ether, carbonyl, (e.g., non-oxo-carbonyl), such as carboxylic acid ester, and the like. The nitrogen may be present as amide, cyano and amino. The polymers set forth in Heller, Biodegradable Polymers in Controlled Drug Delivery: CRC Critical Reviews in Therapeutic Drug Carrier Systems, Vol. 1, CRC Press, Boca Raton, Fla. 1987, pp 39-90, which describes encapsulation for controlled drug delivery, may find use in the present drug delivery systems.

Of additional interest are polymers of hydroxyaliphatic carboxylic acids, either homopolymers or copolymers, and polysaccharides. Polyesters of interest include polymers of D-lacetic acid, L-lacetic acid, racemic lacetic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof, which are biocompatible and may be biodegradable and/or bioerodible.

Some preferred characteristics of the polymers or polymeric materials for use in the present systems may include biocompatibility, compatibility with the therapeutic component, ease of use of the polymer in making the drug delivery systems of the present invention, a half-life in the physiological environment of at least about 6 hours, preferably greater than about one day, not significantly increasing the viscosity of the vitreous, and water insolubility.

The biodegradable polymeric materials, which are included to form the matrix, are desirably subject to enzymatic or hydrolytic instability. Water-soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Also important to controlling the biodegradation of the polymer and hence the extended release profile of the drug delivery systems is the relative average molecular weight of the polymeric composition employed in the present systems.

Different molecular weights of the same or different polymeric compositions may be included in the systems to modulate the release profile. In certain systems, the relative average molecular weight of the polymer will range from about 9 to about 64 kD, usually from about 10 to about 54 kD, and more usually from about 12 to about 45 kD.

In some drug delivery systems, copolymers of glycolic acid and lacetic acid are used, where the rate of biodegradation is controlled by the ratio of glycolic acid to lacetic acid. The most rapidly degraded copolymer has roughly equal amounts of glycolic acid and lacetic acid. Homopolymers, or copolymers having ratios other than equal, are more resistant to degradation. The ratio of glycolic acid to lacetic acid will also affect the brittleness of the system, where a more flexible system or implant is desirable for larger geometries. The % of polylacetic acid in the polylacetic acid polyglycolic acid (PLGA) copolymer can be 0-100%, preferably about 15-85%, more preferably about 35-65%. In some systems, a 50/50 PLGA copolymer is used.

The biodegradable polymer matrix of the present systems may comprise a mixture of two or more biodegradable polymers. For example, the system may comprise a mixture of a first biodegradable polymer and a different second biodegradable polymer. One or more of the biodegradable polymers may have terminal acid groups.

Release of a drug from a bioerodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. It may be understood that the polymeric component of the present systems is associated with the therapeutic component so that the release of the therapeutic component into the eye is by one or more of diffusion, erosion, dissolution, and osmosis. As discussed herein, the matrix of an intraocular drug delivery system may release drug at a rate effective to sustain release of an amount of the sirtuin-activating agent for more than one week after implantation into an eye. In certain systems, therapeutic amounts of the sirtuin-activating agent are released for more than about one month, and even for about twelve months or more. For example, the therapeutic component can be released into the eye for a time period from about ninety days to about one year after the system is placed in the interior of an eye.

The release of the sirtuin-activating agent from the drug delivery systems comprising a biodegradable polymer matrix may include an initial burst of release followed by a gradual increase in the amount of the sirtuin-activating agent released, or the release may include an initial delay in release of the sirtuin-activating agent followed by an increase in release. When the system is substantially completely degraded, the percent of the sirtuin-activating agent that has been released is about one hundred.

It may be desirable to provide a relatively constant rate of release of the therapeutic agent from the drug delivery system over the life of the system. For example, it may be desirable for the sirtuin-activating agent to be released in amounts from about 0.01 μg (microgram) to about 2 μg (microgram) per day for the life of the system. However, the release rate may change to either increase or decrease depending on the formulation of the biodegradable polymer matrix. In addition, the release profile of the sirtuin-activating agent may include one or more linear portions and/or one or more non-linear portions. Preferably, the release rate is greater than zero once the system has begun to degrade or erode.

The drug delivery systems, such as the intraocular implants, may be monolithic, i.e. having the active agent or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the sirtuin-activating agent falls within a narrow window. In addition, the therapeutic component, including the therapeutic agent(s) described herein, may be distributed in a non-homogenous pattern in the matrix. For example, the drug delivery system may include a portion that has a greater concentration of the sirtuin-activating agent relative to a second portion of the system.

The polymeric implants disclosed herein may have a size of between about 5 μm (micro-meter) and about 2 mm (millimeter), or between about 10 μm (micro-meter) and about 1 mm (millimeter) for administration with a needle, greater than 1 mm (millimeter), or greater than 2 mm (millimeter), such as 3 mm (millimeter) or up to 10 mm (millimeter), for administration by surgical implantation. The vitreous chamber in humans is able to accommodate relatively large implants of varying geometries, having lengths of, for example, 1 to 10 mm (millimeter). The implant may be a cylindrical pellet (e.g., a rod) with dimensions of about 2 mm (millimeter)×0.75 mm (millimeter) diameter. Or the implant may be a cylindrical pellet with a length of about 7 mm (millimeter) to about 10 mm (millimeter), and a diameter of about 0.75 mm (millimeter) to about 1.5 mm (millimeter).

The implants may also be at least somewhat flexible so as to facilitate both insertion of the implant in the eye, such as in the vitreous, and accommodation of the implant within the eye. The total weight of the implant is usually about 250-5000 μg (microgram), more preferably about 500-1000 μg (microgram). For example, an implant may be about 500 μg (microgram), or about 1000 μg (microgram). However, larger implants may also be formed and further processed before administration to an eye. In addition, larger implants may be desirable where relatively greater amounts of the sirtuin-activating agent are provided in the implant. For non-human individuals, the dimensions and total weight of the implant(s) may be larger or smaller, depending on the type of individual. For example, humans have a vitreous volume of approximately 3.8 mL (milliliter), compared with approximately 30 mL for horses, and approximately 60-100 mL for elephants. An implant sized for use in a human may be scaled up or down accordingly for other animals, for example, about 8 times larger for an implant for a horse, or about, for example, 26 times larger for an implant for an elephant.

Drug delivery systems can be prepared where the center may be of one material and the surface may have one or more layers of the same or a different composition, where the layers may be cross-linked, or of a different molecular weight, different density or porosity, or the like. For example, where it is desirable to quickly release an initial bolus of sirtuin-activating agent, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The drug delivery systems may be of any geometry including fibers, sheets, films, microspheres, spheres, circular discs, plaques and the like. The upper limit for the system size will be determined by factors such as toleration for the system, size limitations on insertion, ease of handling, and the like. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of about 0.5 μm (micro-meter) to 4 mm (millimeter) in diameter, with comparable volumes for other shaped particles.

The size and form of the system can also be used to control the rate of release, period of treatment, and drug concentration at the site of implantation. For example, larger implants will deliver a proportionately larger dose, but depending on the surface to mass ratio, may have a slower release rate. The particular size and geometry of the system are chosen to suit the site of implantation.

The proportions of therapeutic agent, polymer, and any other modifiers may be empirically determined by formulating several implants, for example, with varying proportions of such ingredients. A USP-approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as by spectrophotometry, HPLC, mass spectroscopy, and the like until the absorbance becomes constant or until greater than 90% of the drug has been released.

In addition to the therapeutic component containing a sirtuin-activating agent, and similar to the compositions described herein, the polymeric drug delivery systems disclosed herein may include an excipient component. The excipient component may be understood to include solubilizing agents, viscosity inducing agents, buffer agents, tonicity agents, preservative agents, and the like.

Additionally, release modulators such as those described in U.S. Pat. No. 5,869,079 may be included in the drug delivery systems. The amount of release modulator employed will be dependent on the desired release profile, the activity of the modulator, and on the release profile of the therapeutic agent in the absence of modulator. Electrolytes such as sodium chloride and potassium chloride may also be included in the systems. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolves more slowly, slowing the exposure of drug particles, and thereby slowing the rate of drug bioerosion.

Various techniques may be employed to produce such drug delivery systems. Useful techniques include, but are not necessarily limited to, solvent evaporation methods, phase separation methods, interfacial methods, molding methods, injection molding methods, extrusion methods, co-extrusion methods, carver press method, die cutting methods, heat compression, combinations thereof and the like.

Specific methods are discussed in U.S. Pat. No. 4,997,652. Extrusion methods may be used to avoid the need for solvents in manufacturing. When using extrusion methods, the polymer and drug are chosen so as to be stable at the temperatures required for manufacturing, usually at least about 85 degrees Celsius (C). Extrusion methods use temperatures of about 25 degrees C. to about 150 degrees C., more preferably about 65 degrees C. to about 130 degrees C. An implant may be produced by bringing the temperature to about 60 degrees C. to about 150 degrees C. for drug/polymer mixing, such as about 130 degrees C., for a time period of about 0 to 1 hour, 0 to 30 minutes, or 5-15 minutes. For example, a time period may be about 10 minutes, preferably about 0 to 5 min. The implants are then extruded at a temperature of about 60 degrees C. to about 130 degrees C., such as about 75 degrees C.

If desired, mixing the sirtuin-activating agent (e.g., resveratrol) with the polymer component may occur before the extrusion step. Additionally, the sirtuin-activating agent and the polymer component may be in a powder form before mixing.

In addition, the implant may be coextruded so that a coating is formed over a core region during the manufacture of the implant.

Compression methods may be used to make the drug delivery systems, and typically yield elements with faster release rates than extrusion methods. Compression methods may use pressures of about 50-150 psi (about 0.345-1034 kPa), more preferably about 70-80 psi (about 482−551.6 kPa), even more preferably about 76 psi (about 524 kPa), and use temperatures of about 0 degrees C. to about 115 degrees C., more preferably about 25 degrees C.

In certain embodiments of the present invention, a method of producing a sustained-release intraocular drug delivery system comprises combining sirtuin-activating agent and a polymeric material to form a drug delivery system suitable for placement in an eye of an individual. The resulting drug delivery system is effective in releasing the sirtuin-activating agent into the eye for extended periods of time. The method may comprise a step of extruding a particulate mixture of the sirtuin-activating agent and the polymeric material to form an extruded composition, such as a filament, sheet, and the like.

When polymeric particles are desired, the method may comprise forming the extruded composition into a population of polymeric particles or a population of implants, as described herein. Such methods may include one or more steps of cutting the extruded composition, milling the extruded composition, and the like.

As discussed herein, the polymeric material may comprise a biodegradable polymer, a non-biodegradable polymer, or a combination thereof. Examples of polymers include each and every one of the polymers and agents identified above.

Another embodiment relates to a method of producing an ophthalmically therapeutic material that comprises a sirtuin-activating agent. In a broad aspect, the method comprises the steps of selecting a sirtuin-activating agent and combining the selected sirtuin-activating agent with a liquid carrier component and/or a polymeric component to form a material suitable for administration to an eye. Or stated differently, a method of producing the present materials may comprise a step of selecting sirtuin-activating agents having a low aqueous humor/vitreous humor concentration ratio and long intravitreal half-life.

The method may further comprise one or more of the following steps, which will typically be used to select the sirtuin-activating agent: administering an sirtuin-activating agent to an eye of a subject and determining the concentration of the sirtuin-activating agent in at least one of the vitreous humor and aqueous humor as a function of time; and administering a sirtuin-activating agent to an eye of a subject and determining at least one of the vitreous half-life and clearance of the sirtuin-activating agent from the posterior chamber of the eye.

Preferably, the sirtuin-activating agents of the present compositions are administered directly to the vitreous chamber of the eye, by means including administration of a solution, suspension, or other means of carrying of crystals or particles of the sirtuin-activating agent, or as part of an intravitreal implant, by, for example, incision or injection.

The vitreous humor contained in the posterior chamber of the eye is a viscous, aqueous substance. Injection of a fluid or suspension of substantially lower viscosity into the posterior segment could therefore result in the presence of two phases or layers of different density within the eye, which in turn can lead to either “pooling” of sirtuin-activating agent particles or floating of the less dense solution. Additionally, a substantially different refractive index between vitreous and the injected or inserted sirtuin-activating agent composition may impair vision. If the injected or inserted material contains a drug in the form of a solid (for example as crystals, particles, or an unsutured implant, or a reservoir), the solid material will fall to the bottom of the eye and remain there until it dissolves.

Intravitreal delivery of therapeutic agents can be achieved by injecting a liquid-containing composition into the vitreous, or by placing polymeric drug delivery systems, such as implants and microparticles, such as microspheres, into the vitreous. Examples of biocompatible implants for placement in the eye have been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; and 6,699,493.

Other routes of administering the therapeutic agents, containing a sirtuin-activating agent, of the present invention to the interior of the eye may include periocular delivery of drugs to a patient. Penetration of drugs directly into the posterior segment of the eye is restricted by the blood-retinal barriers. The blood-retinal barrier is anatomically separated into inner and outer blood barriers. Movement of solutes or drugs into the internal ocular structures from the periocular space is restricted by the retinal pigment epithelium (RPE), the outer blood-retinal barrier. The cells of this structure are joined by zonulae oclludentae intercellular junctions. The RPE is a tight ion-transporting barrier that restricts paracellular transport of solutes across the RPE. The permeability of most compounds across the blood-retinal barriers is very low. Lipophilic compounds, however, such as chloramphenical and benzyl penicillin, can penetrate the blood-retinal barrier achieving appreciable concentrations in the vitreous humor after systemic administration. The lipophilicity of the compound correlates with its rate of penetration and is consistent with passive cellular diffusion. The blood retinal barrier, however, is impermeable to polar or charged compounds in the absence of a transport mechanism.

Additional embodiments of the present invention are related to methods of improving or maintaining vision of an eye of a patient, or at least preventing further loss or deterioration of vision. In general, the methods comprise a step of administering the present ophthalmically therapeutic material to an eye of an individual in need thereof. Administration, such as intravitreal or periocular (or less preferably, topical) administration of the present materials can be effective in treating posterior ocular conditions without significantly affecting the anterior chamber. The present materials may be particularly useful in treating inflammation and edema of the retina. Administration of the present materials are effective in delivering the sirtuin-activating agent to one or more posterior structures of the eye including the uveal tract, the vitreous, the retina, the choroid, the retinal pigment epithelium.

When a syringe apparatus is used to administer the present materials, the apparatus can include an appropriately sized needle, for example, a 27-gauge needle or a 30-gauge needle. Such apparatus can be effectively used to inject the materials into the posterior segment or a periocular region of an eye of a human or animal. The needles may be sufficiently small to provide an opening that self seals after removal of the needle.

The present methods may comprise a single injection into the posterior segment of an eye or may involve repeated injections, for example over periods of time ranging from about one week or about 1 month or about 3 months to about 6 months or about 1 year or about 5 years or longer.

The present materials are preferably administered to patients in a sterile form. For example, the present materials may be sterile when stored. Any routine suitable method of sterilization may be employed to sterilize the materials. For example, the present materials may be sterilized using radiation. Preferably, the sterilization method does not reduce the activity or biological or therapeutic activity of the therapeutic agents of the present systems.

The materials can be sterilized by gamma irradiation. As an example, the drug delivery systems can be sterilized by 2.5 to 4.0 mrad of gamma irradiation. The drug delivery systems can be terminally sterilized in their final primary packaging system including administration device (e.g., syringe applicator). Alternatively, the drug delivery systems can be sterilized alone and then aseptically packaged into an applicator system.

In this case the applicator system can be sterilized by gamma irradiation, ethylene oxide (ETO), heat, or other means. The drug delivery systems can be sterilized by gamma irradiation at low temperatures to improve stability or blanketed with argon, nitrogen or other means to remove oxygen. Beta irradiation or e-beam may also be used to sterilize the implants as well as UV irradiation. The dose of irradiation from any source can be lowered depending on the initial bioburden of the drug delivery systems such that it may be much less than 2.5 to 4.0 mrad. The drug delivery systems may be manufactured under aseptic conditions from sterile starting components. The starting components may be sterilized by heat, irradiation (gamma, beta, UV), ETO or sterile filtration. Semi-solid polymers or solutions of polymers may be sterilized prior to drug delivery system fabrication and sirtuin-activating agent incorporation by sterile filtration of heat. The sterilized polymers can then be used to aseptically produce sterile drug delivery systems.

The compositions can be administered and can prevent further cell loss or cell degeneration. For example, administration, such as intravitreal administration, of the present compositions can result in a decrease in the rate of cell loss and thereby relieve one or more symptoms of an ophthalmic condition. The present compositions can be administered after the patient experiences some symptoms of the ophthalmic conditions associated with cell loss, such as retinal ganglion cell degeneration. For example, the patient may already have experienced a loss of a portion of retinal ganglion cells and thus has reported with decreased visual acuity or other symptoms associated with that loss or decreased function. Administration of the present compositions can prevent further loss or degeneration of the remaining retinal ganglion cells. In addition, the present compositions can prevent or reduce further degeneration of injured or dying retinal ganglion cells. Typically, the administration of the present composition preserves the function of the eye at the time of administration. However, it is also possible that the administration may improve vision by allowing the surviving retinal ganglion cells to enhance their function and compensate for the degenerated retinal ganglion cells. For example, the surviving retinal ganglion cells may undergo enhanced axonal or dendritic growth to provide physiological activity that was once previously provided by the injured or dead retinal ganglion cells. In certain embodiments, the present compositions are administered to a patient before there is at least 10% retinal ganglion cell loss, or before there is a loss of 20% of the retinal ganglion cells, or a loss of 40% of the retinal ganglion cells, or a loss of 80% of the retinal ganglion cells.

Administration of the present composition alleviates or treats one or more symptoms of an ophthalmic condition. For example, the present compositions can reduce a symptom by at least 10%, such as by at least 20%, or by at least 40%, or by at least 80%. The reduction can be determined subjectively by the patient's own perception of the symptom using standard assessment scales, or the reduction can be determined objectively by a physician or other diagnostician who can measure and quantify the change in the symptom. For example, a patient who has experienced a 20% field of view loss may be treated with the present compositions. A physican can determine whether the vision loss remains stable, improves, or continues to increase. Administration of the present compositions can reduce further vision loss or can improve (e.g., decrease) the amount of vision loss. Thus, the therapeutic effects obtained with the present compositions and methods can be readily determined using conventional techniques and other techniques.

In another aspect of the invention, kits for treating an ocular condition of the eye are provided, comprising: a) a container, such as a syringe or other applicator, comprising a sirtuin-activating agent as herein described; and b) instructions for use. Instructions may include steps of how to handle the material, how to insert the material into an ocular region, and what to expect from using the material. The container may contain a single dose of the sirtuin-activating agent.

In view of the disclosure herein, an embodiment of the present invention can be understood to be an intraocular biodegradable implant. The intraocular biodegradable implant is an extruded element comprising resveratrol or other sirtuin-activating agents and a biodegradable polymer, such as PLGA. The implant degrades when placed in the vitreous of an eye to release the resveratrol in neuroprotecting amounts to reduce neurodegeneration or death of retinal ganglion cells, and thereby ameliorate or reduce one or more symptoms of an ophthalmic condition being treated. The implant is placed in the eye to treat degenerative conditions, such as glaucoma, macular degeneration, and diabetic retinopathy. The implant provides local delivery of resveratrol or other sirtuin-activating agent with minimal systemic exposure, continuous and high level exposure of the resveratrol at the target site, and reduced unwanted drug-drug interactions when ocular administration and systemic administration are used on a patent.

In further embodiments, other sirtuin-activating agents, including polyphenolic compounds that activate sirtuin, can be provided in the extruded implants described above.

### EXAMPLES

### Example 1

### Sirtuin-Activating Agent Implant

Biodegradable drug delivery systems may be made by combining a sirtuin-activating agent with a biodegradable polymer composition in a stainless steel mortar. The combination can then be mixed via a Turbula shaker set at 96 RPM for 15 minutes. The powder blend is scraped off the wall of the mortar and then remixed for an additional 15 minutes. The mixed powder blend may be heated to a semi-molten state at specified temperature for a total of 30 minutes, forming a polymer/drug melt.

Rods may be manufactured by pelletizing the polymer/drug melt using a 9-gauge polytetrafluoroethylene (PTFE) tubing, loading the pellet into the barrel and extruding the material at the specified core extrusion temperature into filaments. The filaments may then be cut into about 1 mg size implants or drug delivery systems. The rods may have dimensions of about 2 mm long×0.72 mm diameter. The rod implants may weigh between about 900 μg (microgram) and 1100 μg (microgram).

Wafers may be formed by flattening the polymer melt with a Carver press at a specified temperature and cutting the flattened material into wafers, each weighing about 1 mg. The wafers may have a-diameter of about 2.5 mm and a thickness of about 0.13 mm. The wafer implants may weigh between about 900 μg (microgram) and 1100 μg (microgram).

In-vitro release testing can be performed on each lot of implant (rod, wafer, or other form). Each implant may be placed into a 24 mL screw cap vial with 10 mL of Phosphate Buffered Saline solution at 37° C. and 1 mL aliquots may be removed and replaced with equal volume of fresh medium on day 1, 4, 7, 14, 28, and every two weeks thereafter.

Drug assays may be performed by HPLC, which consists of a Waters 2690 Separation Module (or 2696), and a Waters 2996 Photodiode Array Detector. An Ultrasphere, C-18 (2), 5 μm (micro-meter); 4.6×150 mm column heated at 30° C. can be used for separation and the detector can be set at 264 nm. The mobile phase can be (10:90) MeOH-buffered mobile phase with a flow rate of 1 mL/min and a total run time of 12 min per sample. The buffered mobile phase may comprise (68:0.75:0.25:31) 13 mM 1-Heptane Sulfonic Acid, sodium salt-glacial acetic acid-triethylamine-Methanol. The release rates can be determined by calculating the amount of drug being released in a given volume of medium over time in μg (microgram)/day.

The polymers chosen for the implants can be obtained from Boehringer Ingelheim or Purac America, for example. Examples of polymers include: RG502, RG752, R202H, R203 and R206, and Purac PDLG (50/50). RG502 is (50:50) poly (D,L-lactide-co-glycolide), RG752 is (75:25) poly (D,L-lactide-co-glycolide), R202H is 100% poly (D, L-lactide) with acid end group or terminal acid groups, R203 and R206 are both 100% poly (D, L-lactide). Purac PDLG (50/50) is (50:50) poly (D,L-lactide-co-glycolide). The inherent viscosity of RG502, RG752, R202H, R203, R206, and Purac PDLG are 0.2, 0.2, 0.2, 0.3, 1.0, and 0.2 dL/g, respectively. The average molecular weight of RG502, RG752, R202H, R203, R206, and Purac PDLG are, 11700, 11200, 6500, 14000, 63300, and 9700 daltons, respectively.

### Example 2

### Manufacture of Double Extrusion Sirtuin-Activating Agent Implant

Double extrusion processes may also be used for the manufacture of sirtuin-activating agent implants. Such implants can be made as follows, and as set forth in U.S. Patent Publication No. 20050048099, hereby incorporated by reference herein.

For example, a biodegradable polymer, such as a PLGA polymer or any of the polymers set forth herein, can be milled using a vibratory feeder and grinding nozzle to form particles of the biodegradable polymer. The particles can be sorted or formed to produce a population of particles having a pre-determined size, such as a diameter of about 20 μm.

Particles of one or more sirtuin-activating agents can be combined with the biodegradable polymer particles to form a blended mixture. The blended mixture can then be extruded using an extrusion device, such as a Haake Twin Screw Extruder, to form an extruded composition or product, such as an extruded filament. The extruded product can then be pellitized. The pelletized extruded product can then undergo a second extrusion step to produce a double-extruded element comprising a biodegradable polymer and at least one sirtuin-activating agent. The double extruded element can be in the form of an intraocular implant, or it can be in the form of a larger product, such as a filament, which can be processed to form implants sized for intraocular placement in an eye of a patient, such as in the vitreous of an eye.

### Example 3

### Treatment of Macular Edema with a Resveratrol Implant

A 58-year-old man may be diagnosed with cystic macular edema. The man is treated by administration of a biodegradable drug delivery system administered to each eye of the patient. A 2-mg intravitreal implant containing about 1000 μg (microgram) of PLGA and about 1000 μg (microgram) of resveratrol (trans isomer) is placed in his left eye at a location that does not interfere with the man's vision. A similar or smaller implant is administered subconjunctivally to the patient's right eye. A more rapid reduction in retinal thickness in the right eye may occur due to the location of the implant and the activity of the resveratrol. After about 3 months from the surgery, a normal appearing retina and a reduction in optic nerve degeneration indicates successful treatment with the resveratrol implant. One week after administration of the implant, an intraocular pressure that is similar to the pressure before the placement of the implant in the eye can be reflective of no apparent side effects associated with the implant.

### Example 4

### Treatment of ARMD with a Sirtuin-Activating Agent Composition

A 62-year-old woman with wet age-related macular degeneration may be treated with an intravitreal injection of 100 μL (microliter) of a hyaluronic acid solution containing about 1000 μg (microgram) of resveratrol (trans isomer) crystals in suspension. Within one month following administration the patient may then exhibit an acceptable reduction in the rate of neovascularization and related inflammation. The patient may then report an overall improvement in quality of life.

### Example 5

### Neuroprotective Effects of a Sirtuin-Activating Agent on Retinal Ganglion Cells

The known rat optic nerve crush model can be used to induce injury to retinal ganglion cells. A sirtuin-activating agent is administered to the rat after the optic nerve injury in one or more doses. The agent can be administered intraocularly, such as by placement of an implant or other sirtuin-activating agent-containing composition in the vitreous of an eye. After a desired amount of time, such as at least one week, the eyes can be removed and histologically processed. Retinal ganglion cell counts can be performed on stained histological sections. Increased cell counts of animals receiving the sirtuin-activating agent compared to vehicle treated controls, such as animals administered saline or other drug-free composition, indicates a protective effect of the sirtuin-activating agent on the retinal ganglion cells. A correlation between the number of surviving retinal ganglion cells and the dose of the sirtuin-activating agent indicates a dose dependent response of the neuroprotective effects of the agent.

### Example 6

### Neuroprotective Effects of a Polyphenolic Sirtuin-Activating Agent on Retinal Ganglion Cells

Example 5 can be repeated using a polyphenolic sirtuin-activating agent. For example, an biodegradable intraocular implant produced in accordance with the method of example 1 or example 2 can include a polyphenolic sirtuin-activating agent having the following formula (Formula I):

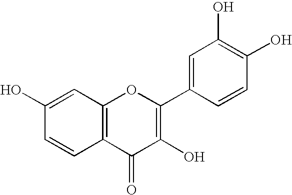

Formula I is the formula for fisetin.

Or the implant can include a polyphenolic sirtuin-activating agent having the following formula (Formula II):

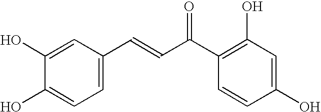

Formula II is the formula for butein.

### Example 7

### Neuroprotective Effects of Resveratrol on Retinal Ganglion Cells

Example 5 can be repeated using the trans isomer of resveratrol as the neuroprotective agent. For example, an implant can comprise a polyphenolic sirtuin-activating agent having the following formula (Formula III):

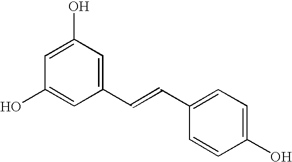

Formula III is the formula for resveratrol.

### Example 8

### Retinal Ganglion Cell Survival after Administration of Resveratrol

Sprague Dawley rats weighing 300-350 g were anesthetized with a mixture of ketamine (50 mg/kg), and xylazine (0.5 mg/kg). Lateral canthotomy was performed in the right eyes; an incision was made in the superior conjunctiva adjacent to the rectus muscle. This was followed by a blunt dissection until the optic nerve was exposed. A partial crush was applied to the optic nerve for 30 seconds, 3 to 4 mm distal from the globe avoiding the retinal blood supply, using calibrated cross acting forceps. Resveratrol (trans isomer) at different doses were given once by intraperitoneal injection, immediately after optic nerve injury. Control animals received phosphate buffered saline (PBS) vehicle. The experiment was terminated 12 days later.

At the end 12 days, the retinal ganglion cells were labeled by retrograde transport of dextran tetramethyl rhodamine (DTMR, 3000 MW). The optic nerve was completely transected at about 2 to 3 mm proximal to the globe and the dye was applied at the exposed optic nerve. Twenty four hours later, the rats were euthanized, eyes enucleated and fixed with 4% paraformaldehyde. The retinas were then removed and whole-mounted. Fluorescently-labeled ganglion cells were counted in 8 to 16 regions in the four quadrants of whole mounted retina. Cell counts in vehicle-treated retinas from injured optic nerves were normalized to one and the increase in cell survival by drugs (treated) was calculated in relation to the vehicle (control) treated group.

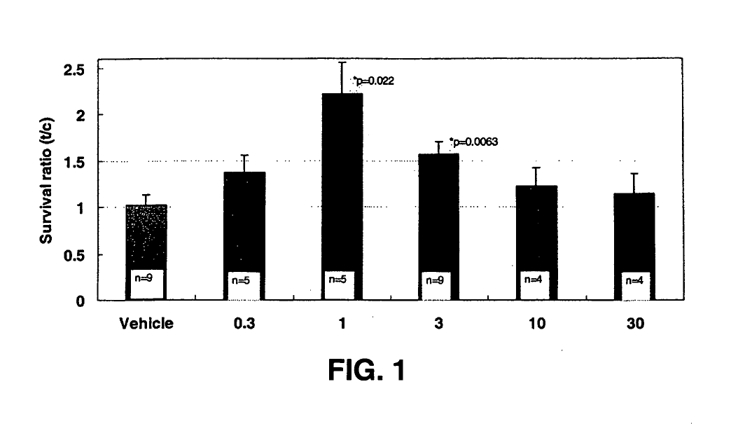

The results of these experiments are illustrated in FIG. 1. FIG. 1 is a graph of retinal ganglion cell survival ratio (treated/control; t/c) as a function of resveratrol dose (mg/kg). In this procedure, resveratrol was administered intraperitoneally immediately after injury and at 5 hours post injury. As shown in FIG. 1, resveratrol at intraperitoneal doses of 1 mg/kg and 3 mg/kg resulted in a significant increase in the retinal ganglion cell survival ratio. Increases in the ratio were also observed at 0.3 mg/kg, 10 mg/kg, and 30 mg/kg. These results demonstrate that resveratrol can provide desirable neuroprotective effects to ocular cells.

All references, articles, publications, and patents, and patent applications cited herein are incorporated by reference in their entireties.

While this invention has been described with respect to various specific examples and embodiments, it is to be understood that the invention is not limited thereto and that it can be variously practiced within the scope of the following claims.

1. An intraocular implant, comprising:
a sirtuin-activating agent; and
a bioerodible polymer matrix that releases the sirtuin-activating agent at a rate effective to sustain release of an amount of the sirtuin-activating agent from the implant for at least about one week after the implant is placed in an eye.

2. The implant of claim 1, wherein the sirtuin-activating agent is selected from the group consisting of flavones, stilbenes, flavanones, isoflavones, catechins, chalcones, tannins, anthocyanidins, analogs thereof, and derivatives thereof.

3. The implant of claim 1, wherein the sirtuin-activating agent is selected from the group consisting of resveratrol, butein, piceatannol, isoliquiritgenin, fisetin, luteolin, 3,6,3′4′-tetrahydroxyflavone, quercetin, analogs thereof, and derivatives thereof.

4. The implant of claim 1, wherein the bioerodible polymer matrix is selected from the group consisting of poly (lactide-co-glycolide) polymer (PLGA), poly-lacetic acid (PLA), poly-glycolic acid (PGA), polyesters, poly (ortho ester), poly (phosphazine), poly (phosphate ester), poly (D,L-lactide-co-glycolide), polyesters, polycaprolactones, gelatin, and collagen, and derivatives and combinations thereof.

5. The implant of claim 1, further comprising an additional ophthalmically acceptable therapeutic agent.

6. The implant of claim 1, wherein the sirtuin-activating agent is dispersed within the bioerodible polymer matrix.

7. The implant of claim 1, wherein the bioerodible polymer matrix comprises a poly (lactide-co-glycolide).

8. The implant of claim 1, wherein the bioerodible polymer matrix comprises a poly (D,L-lactide-co-glycolide).

9. The implant of claim 1, wherein the bioerodible polymer matrix releases sirtuin-activating agent at a rate effective to sustain release of an amount of the sirtuin-activating agent from the implant for more than one month from the time the implant is placed in the vitreous of the eye.

10. The implant of claim 1, wherein the sirtuin-activating agent is resveratrol, and the matrix releases resveratrol at a rate effective to sustain release of a therapeutically effective amount of the resveratrol for a time from about two months to about six months.

11. The implant of claim 1, wherein the implant is structured to be placed in the vitreous of the eye.

12. The implant of claim 1, wherein the sirtuin-activating agent is resveratrol provided in an amount from about 40% by weight to about 70% by weight of the implant, and the biodegradable polymer matrix comprises a poly (lactide-co-glycolide) in an amount from about 30% by weight to about 60% by weight of the implant.

13. The implant of claim 1, formed as a rod, a wafer, or a particle.

14. The implant of claim 1, formed by an extrusion process.

15. The implant of claim 1, wherein the sirtuin-activating agent contains particles comprising resveratrol in solid form.

16.-29. (canceled)
